(19) United States
(12) Patent Application Publication
(10) Pub. No.: US 2006/0121622 A1
(43) Pub. Date: Jun. 8, 2006

(54) Method for determination of organic hydroperoxides

(76) Inventors: Robert B. Crippen, Willow Grove, PA (US); Robert W. Emblidge, Ivyland, PA (US)

Correspondence Address: Robert A. Koons, Jr.;Buchanan Ingersoll PC, 14th Floor, 1835 Market Street, Philadelphia, PA, 19103-2985, US

(21) Appl. No.: 11/001,719

(22) Filed: Dec. 2, 2004

(51) Int. Cl. G01N 33/00

(52) U.S. Cl. 436/135

(57) ABSTRACT

The concentration of an organic hydroperoxide in an aqueous stream is determined by derivatizing the organic hydroperoxide to the corresponding alcohol by addition of acetic acid and potassium iodide. The corresponding alcohol is then quantified by headspace gas chromatography and correlated to a concentration of the organic hydroperoxide in the aqueous stream.

### FIELD OF THE INVENTION

The invention relates generally to the field of analytical methods for the determination of organic contaminants in wastewater streams. More particularly, the present invention relates to gas chromatographic methods for the determination of organic contaminants in aqueous streams.

### BACKGROUND OF THE INVENTION

In industrial processes a continuing focus of environmental concern is the organic content of waste water streams. Of particular concern in a number of processes are organic hydroperoxides, which are either used as a catalyst or reagent in the process, or are produced as a by-product of the process.

A number of methods for the detection of hydrogen peroxide, as well as organic hydroperoxides in solution are documented in the literature. For example, an iodometric method involves reaction of the hydroperoxide with an iodide ion, such as potassium iodide or sodium iodide, and acetic acid, producing iodine, which can subsequently be estimated calorimetrically by titration. Another titrametric method utilizes stannous chloride to reduce the hydroperoxide. The excess stannous chloride is subsequently determined by titration with ferric ion.

The drawback of titrametric methods is that they cannot differentiate between different hydroperoxides without additional procedures. In addition, such methods may be of questionable accuracy.

Weinstein-Lloyd, et al. disclose the continuous measurement of atmospheric hydrogen peroxide, as well as methyl hydroperoxide and hydroxymethyl hydroperoxide using both peroxidase/p-hydroxy phenylacetic acid and ferrous sulfate/benzoic acid reagents to derivatize the hydroperoxides, “Measurements of Peroxides and Related Species During the 1995 Summer Intensive of the Southern Oxidants Study in Nashville, Tenn.”, Weinstein-Lloyd, et al. BNL-64934-98/08-Rev. Other methods have used HPLC followed by post-column derivitization to quantify atmospheric organic hydroperoxides. However, none of these references address the determination of the concentration of organic hydroperoxides in aqueous solutions.

Therefore, there remains a need in the art for an accurate method for the determination of organic hydroperoxides in water streams that can differentiate and quantify various organic hydroperoxides.

### SUMMARY OF THE INVENTION

The present invention provides a method for determining the concentration of an organic hydroperoxide in an aqueous stream. The method comprises the steps of providing an aliquot from an aqueous stream containing at least one organic hydroperoxide, adding acetic acid to the aliquot, adding potassium iodide to the aliquot, and the sealing said aliquot in a sealable sample container. The aliquot is heated in the sealable sample container at a temperature above about 65° C. and below about 100° C. for a time sufficient to convert the organic hydroperoxide into at an alcohol and equilibrate the liquid and gas phases in the sample container. A gaseous sample of the aliquot is then extracted from the sample container, and analyzed by gas chromatography to obtain a quantified value of alcohol in said gaseous sample. This value is then correlated to a concentration of organic hydroperoxide in the aqueous stream.

### BRIEF DESCRIPTION OF THE DRAWINGS

FIG. 1: Illustrates a graph of recovery of dimethylphenyl carbinol (DMPC) from a sample of fresh water.

FIG. 2: Illustrates a graph of recovery of dimethylphenyl carbinol (DMPC) from a sample of water having a low concentration of salt.

FIG. 3: Illustrates a graph of recovery of dimethylphenyl carbinol (DMPC) from a sample of water having a high concentration of salt.

### DETAILED DESCRIPTION OF THE INVENTION

The present invention is a method for the determination of organic hydroperoxides in a water stream using headspace gas chromatography. The method makes use of pre-analysis derivatization of the hydroperoxide to form a corresponding alcohol, which can then be quantified using standard headspace gas chromatography techniques. Once quantified, the alcohol can then be correlated to a concentration of the organic hydroperoxide in the water stream.

The method takes advantage of the following chemistry to derivatize the hydroperoxide:
R—O—O—H+2R′CO₂H+3KI→R—OH+2R′CO₂K+KI+H₂O+I₂
where R is an organic species and R′ is hydrogen, —CH₃ or CH₂CH₃.

The derivatization is preferably performed by adding a sample aliquot of the water stream to be analyzed directly to a headspace gas chromatography (GC) vial, adding acetic acid and potassium iodide directly to the vial and diluting the sample to volume. The headspace GC vial is then sealed and heated at 65 to 100° C., preferably about 80° C., for an appropriate aging period to decompose the organic hydroperoxides present in the aliquot to the corresponding alcohols and equilibrate the liquid and gas phases in the sample vial. The preferred heating period is at least 15 minutes.

The sample aliquot of the water stream used for the analysis is preferably about 1 mL. To this volume is added about 0.05 to about 0.15 mL of acetic acid, and up to 0.25 mL of potassium iodide solution. The potassium iodide solution is preferably a 50 percent by weight solution in water. In this embodiment the total sample volume is made up to 2 mL by adding 0.60 to 0.85 mL of water.

Although the preferred embodiment makes use of acetic acid and potassium iodide, those of ordinary skill in the art will recognize that any water soluble carboxylic acid and iodide ion source will be functional in the current method.

After the appropriate aging period has elapsed, a gaseous sample is withdrawn from the headspace of the sample vial for analysis by gas chromatography. The alcohols present in the gaseous sample are quantified and then correlated to a concentration of the corresponding hydroperoxides in the water sample using methods known in the art.

In a preferred embodiment, in addition to the derivatized samples of the water stream as prepared above, an “as is” sample of the water stream is prepared. A 2 mL sample aliquot is sealed in a headspace GC vial and heated at 65 to 100° C. for the same aging period as the derivatized sample, preferably at least 15 minutes, and at 80° C. The analysis of a gaseous sample of the “as is” sample is performed by gas chromatography using the same conditions as those used for the derivatized samples. This analysis allows any native alcohols in the water stream to be accounted for and the quantity of alcohols calculated for the derivatized samples to be corrected accordingly.

In some instances samples from a process stream will need to be diluted with fresh water prior to analysis. This is often the case with water streams that contain significant amounts of dissolved salts, which may interfere with gas chromatographic analysis in general, and headspace analysis in particular. The dilution ratio used can range from 1:10 to 1:100, and will depend on the organic hydroperoxide be tested for, its concentration in the water stream and the quantity of salts in the water stream.

The method according to the current invention will now be explained more fully with reference to the following examples.

### EXAMPLE 1

A common organic hydroperoxide found in the wastewater effluent streams of phenol plants is cumene hydroperoxide (CHP), which is the precursor to phenol in the process. On treatment with acetic acid and potassium iodide, cumene hydroperoxide decomposes to dimethylphenyl carbinol (DMPC, a.k.a. dimethylbenzyl alcohol or DMBA). A method according to the present invention for the determination of CHP in wastewater streams was developed using the following procedure.

A series of samples of DMPC in water were prepared at various dilutions to determine the percent recovery of DMPC from an aqueous solution by headspace gas chromatography. Samples were prepared in deionized water, as well as water containing salts at both low and high concentration. The salt solutions were used to mimic the quality of the wastewater streams commonly found in phenol plants. Samples were prepared both with and without the reagents that are used to derivatize CHP, i.e. acetic acid and potassium iodide. Table 1 shows the data from these trials.

TABLE 1

| | WATER | | | LOW SALT | | | HIGH SALT | | |
|---|---|---|---|---|---|---|---|---|---|
| | THEORY | AS IS | DERIV.* | THEORY | AS IS | DERIV.* | THEORY | AS IS | DERIV.* |
| SAMPLE | PPM | PPM | PPM | PPM | PPM | PPM | PPM | PPM | PPM |
| Stock | 111 | 97 | 91 | 108 | 107 | 94 | 106 | 122 | 100 |
| Stock | 111 | 99 | 94 | 108 | 108 | 89 | 106 | 125 | 118 |
| 50% | 55.5 | 50 | 45 | 54 | 50 | 43 | 53 | 56 | 46 |
| 25% | 27.8 | 24 | 22 | 27 | 24 | 21 | 26.5 | 28 | 23 |
| 1% | 1.1 | 1.4 | 1.3 | 1.1 | 1 | 0.9 | 1.1 | 1.3 | 0.9 |

*These samples were run with acetic acid and potassium iodide to simulate actual CHP samples

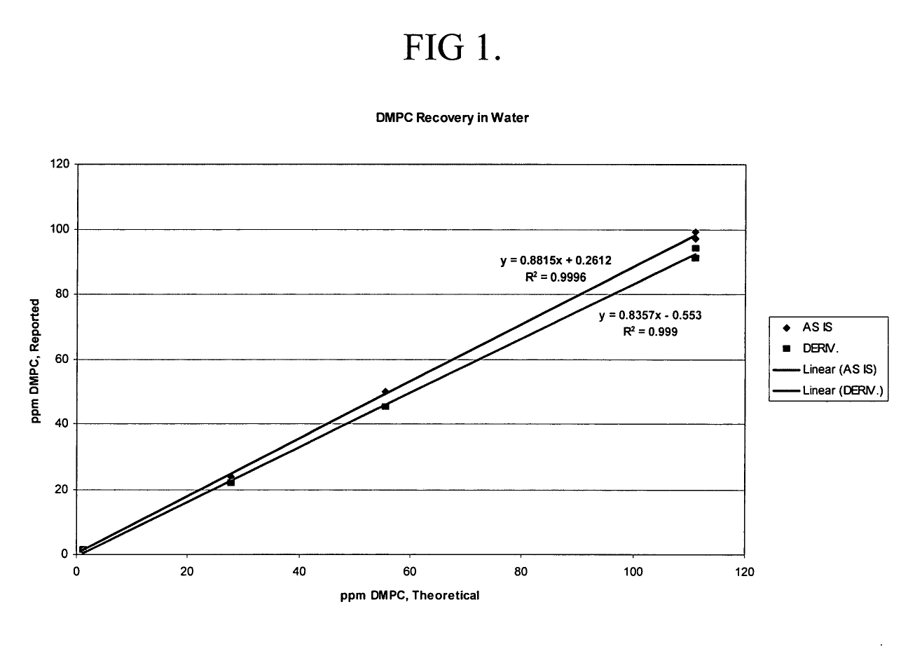

For each sample: water, low salt and high salt, the first column shows the theoretical amount of DMPC in parts per million (ppm) that should be detected in the solution. The second and third columns show the amount actually detected in ppm by headspace GC. From this information, a percentage recovery of DMPC from and aqueous sample can be determined. FIGS. 1 through 3 show the recovery of DMPC versus the theoretical that should be found. In each Figure, the slope of the regression line is the percentage recovery.

Next, stock solutions of CHP in water were derivatized to DMPC according to the method of the current invention and analyzed by the same headspace GC method as was used for the DMPC samples in Table 1. Again, the derivatized samples were run in deionized water, as well as salt solutions of low and high concentration. The results are shown in Tables 2, 3 and 4.

Table 2 shows the CHP results for fresh water. In each case, the second column shows the theoretical amount of CHP (DMPC) that should be found in ppm based on the concentration of CHP in the solution as prepared. The third column shows the quantity actually found in ppm, and the fourth column shows a percent recovery based on the amount of CHP (DMPC) actually found. The fifth column in Table 2 shows a corrected quantity of CHP (DMPC) found based on the DMPC recoveries calculated for the trials in Table 1.

TABLE 2
Fresh Water

| | THEORY | FOUND | | CORRECTED | CORRECTED % |
|---|---|---|---|---|---|
| SAMPLE | PPM | PPM | % RECOVERY | FOUND PPM | RECOVERY |
| Stock | 511 | 360 | 70 | 431.4 | 84.4 |
| Stock | 511 | 348 | 68 | 417.1 | 81.6 |
| 50% | 255.5 | 161 | 63 | 193.3 | 75.6 |
| 50% | 255.5 | 173 | 68 | 207.6 | 81.3 |
| 25% | 127.75 | 82 | 64 | 98.7 | 77.3 |
| 25% | 127.75 | 82 | 64 | 98.7 | 77.3 |
| 1% | 5.11 | 3 | 59 | 4.1 | 81.1 |
| 1% | 5.11 | 3 | 59 | 4.1 | 81.1 |

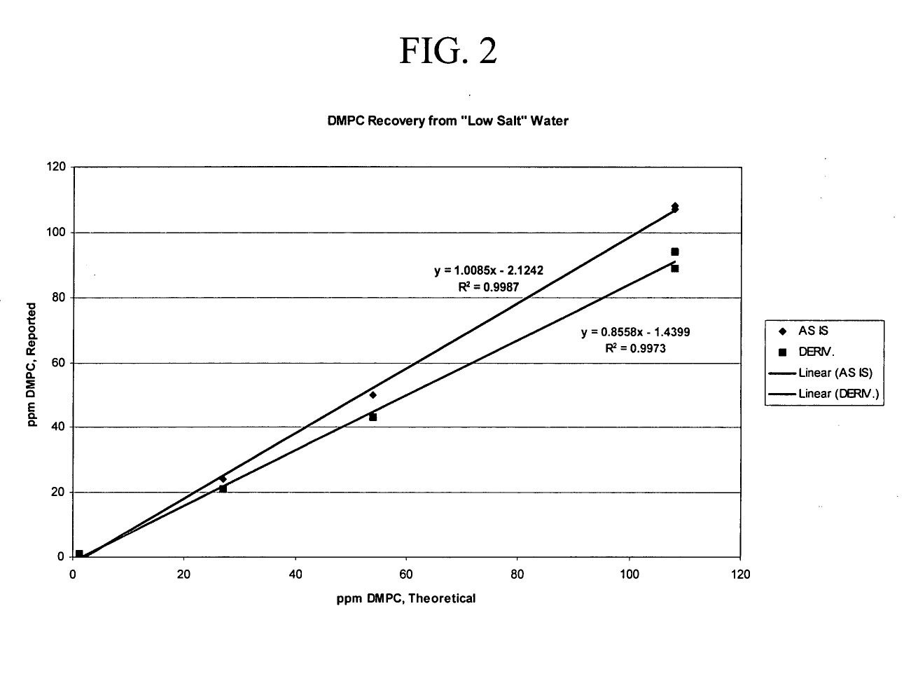

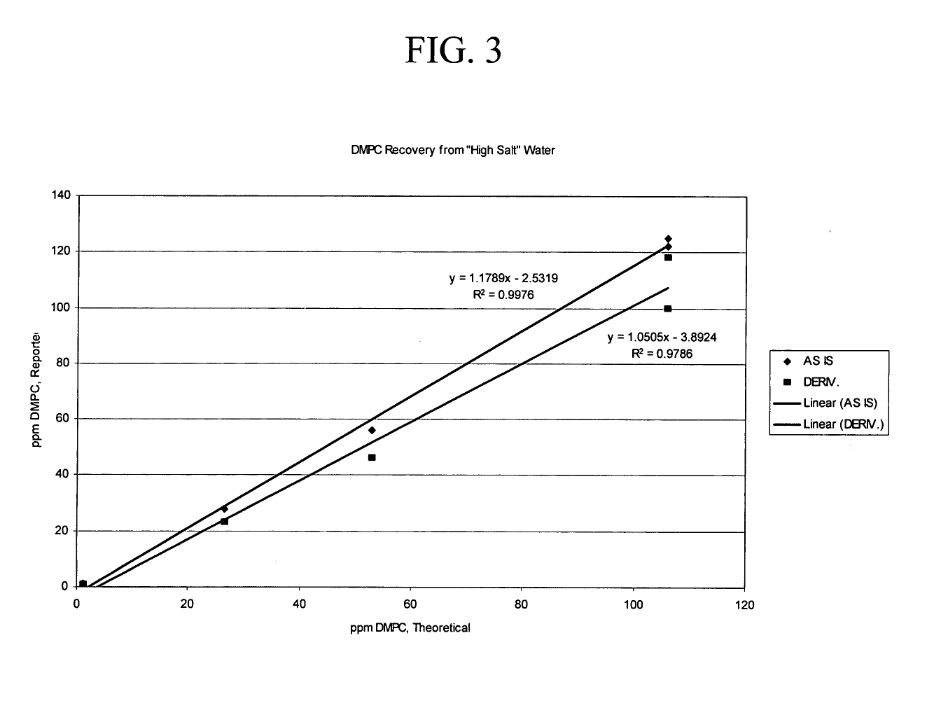

For example, using the equation shown in FIG. 1 for fresh water, y=0.8357×−0.553, the actual quantity of CHP (DMPC) detected for the first stock solution is (360/0.8357)+0.553=431.4. From this number, the corrected percent recovery can be calculated. Similar results are obtained in Tables 3 and 4 using the appropriate equations from FIGS. 2 and 3.

TABLE 3
Low Salt

| | THEORY | FOUND | | CORRECTED | CORRECTED |
|---|---|---|---|---|---|
| SAMPLE | PPM | PPM | % RECOVERY | FOUND PPM | % RECOVERY |
| Stock | 511 | 333 | 65 | 390.5 | 76.4 |
| Stock | 511 | 363 | 71 | 425.6 | 83.3 |
| 50% | 255.5 | 169 | 66 | 198.9 | 77.9 |
| 50% | 255.5 | 165 | 64 | 194.2 | 76.0 |
| 25% | 127.75 | 79 | 62 | 93.8 | 73.4 |
| 1% | 5.11 | 3 | 59 | 4.9 | 96.8 |

TABLE 4
High Salt

| | THEORY | FOUND | | CORRECTED | CORRECTED |
|---|---|---|---|---|---|
| SAMPLE | PPM | PPM | % RECOVERY | FOUND PPM | % RECOVERY |
| Stock | 514 | 350 | 68 | 337.1 | 65.6 |
| Stock | 514 | 369 | 72 | 355.2 | 69.1 |
| 50% | 257 | 172 | 67 | 167.6 | 65.2 |
| 25% | 128.5 | 85 | 66 | 84.8 | 66.0 |
| 1% | 5.14 | 3 | 59 | 6.7 | 131.3 |

From this information it is possible to derive a calculation to determine an unknown quantity of CHP present in a water stream by headspace GC.

As can be seen by comparing the results in Tables 2, 3 and 4 the presence of salts in the water stream has a negative impact on the recovery of the alcohol and thus on the quantification of the corresponding hydroperoxide.

### EXAMPLE 2

Various process streams were analyzed for cumene hydroperoxide (CHP) and methyl hydroperoxide using the method according to the current invention. All of the samples, which contained significant quantities of salts, were diluted with water at a 1:10 ratio prior to preparation and analysis. CHIP was detected as DMPC and MHP was detected as methanol (MeOH). Tables 5 through 10 show the results of analyses performed on various process streams from a phenol plant. Native methanol and DMPC are determined from un-derivatized “as is” samples. A second set of samples were derivatized according to the current invention by adding to a 1 mL aliquot of the diluted water stream, 0.10 mL of acetic acid, 0.15 mL of 50% potassium iodide in water and 0.75 mL of water. Actual MHP and CHP concentrations were calculated from the difference in methanol and DMPC concentrations between the “as is” samples and the derivatized samples, corrected for dilution and percent recovery.

TABLE 5

| | Day 1 | Day 2 | Day 3 | Day 4 | Day 5 |
|---|---|---|---|---|---|
| Component | Conc. ppm | Conc. ppm | Conc. ppm | Conc. ppm | Conc. ppm |
| MeOH (as is) | 527.69 | 564.93 | 623.61 | 607.87 | 588.55 |
| MeOH (deriv.) | 604.88 | 638.79 | 652.37 | 631.11 | 661.57 |
| DMPC (as is) | 508.32 | 613.18 | 666.44 | 685.66 | 604.76 |
| DMPC (deriv.) | 565.92 | 649.16 | 604.15 | 588.69 | 643.69 |
| MHP (calc.) | 115.79 | 110.79 | 43.14 | 34.86 | 109.53 |
| CHP (calc.) | 89.11 | 55.19 | 0.00 | 0.00 | 59.82 |

TABLE 6

| | Day 1 | Day 2 | Day 3 | Day 4 | Day 5 |
|---|---|---|---|---|---|
| Component | Conc. ppm | Conc. ppm | Conc. ppm | Conc. ppm | Conc. ppm |
| MeOH (as is) | 612.81 | 647.73 | 662.29 | 715.06 | 424.68 |
| MeOH (deriv.) | 658.08 | 688.65 | 721.8 | 739.94 | 491.92 |
| DMPC (as is) | 697.27 | 646.07 | 724.98 | 863.76 | 475.26 |
| DMPC (deriv.) | 650.56 | 624.09 | 671.8 | 761.1 | 525.07 |
| MHP (calc.) | 0.00 | 0.00 | 0.00 | 0.00 | 0.00 |
| CHP (calc.) | 0.00 | 0.00 | 0.00 | 1.54 | 0.00 |

TABLE 7

| | Day 1 | Day 2 | Day 3 | Day 4 | Day 5 |
|---|---|---|---|---|---|
| Component | Conc. ppm | Conc. ppm | Conc. ppm | Conc. ppm | Conc. ppm |
| MeOH (as is) | 201.25 | 223.77 | 226.64 | 213.31 | 200.07 |
| MeOH (deriv.) | 254.97 | 265.07 | 242.63 | 209.31 | 1968.43 |
| DMPC (as is) | 264.88 | 243.84 | 250.28 | 227.74 | 211.7 |
| DMPC (deriv.) | 290.62 | 242.99 | 247.62 | 227.25 | 63.23 |
| MHP (calc.) | 80.58 | 61.95 | 23.99 | 0.00 | 2652.54 |
| CHP (calc.) | 39.13 | 0.00 | 0.00 | 0.00 | 0.00 |

TABLE 8

| | Day 1 | Day 2 | Day 3 | Day 4 |
|---|---|---|---|---|
| Component | Conc. ppm | Conc. ppm | Conc. ppm | Conc. ppm |
| MeOH (as is) | 1710.95 | 1628.74 | 1511.27 | 1575.68 |
| MeOH (deriv.) | 2728.87 | 2784.63 | 2642.23 | 2754.52 |
| DMPC (as is) | 881.46 | 967.83 | 933.7 | 1069.39 |
| DMPC (deriv.) | 2754.58 | 3071.67 | 3008.05 | 3091.61 |
| MHP (calc.) | 1526.88 | 1733.84 | 1696.44 | 1768.26 |
| CHP (calc.) | 2937.33 | 3299.29 | 3253.03 | 3171.24 |

TABLE 9

| | Day 1 | Day 2 | Day 3 | Day 4 |
|---|---|---|---|---|
| Component | Conc. ppm | Conc. ppm | Conc. ppm | Conc. ppm |
| MeOH (as is) | 1975.3 | 1786.42 | 1745.12 | 1598.75 |
| MeOH (deriv.) | 2105.52 | 1914.52 | 1897.23 | 1839.86 |
| DMPC (as is) | 1949.55 | 1849.29 | 1942.45 | 1797.07 |
| DMPC (deriv.) | 2347.85 | 2627.45 | 2757.34 | 2660.7 |
| MHP (calc.) | 195.33 | 192.15 | 228.17 | 361.67 |
| CHP (calc.) | 623.61 | 1219.54 | 1277.16 | 1353.63 |

TABLE 10

| | Day 1 | Day 2 | Day 3 | Day 4 |
|---|---|---|---|---|
| Component | Conc. ppm | Conc. ppm | Conc. ppm | Conc. ppm |
| MeOH (as is) | 5057.95 | 5302.72 | 5151.97 | 5287.79 |
| MeOH (deriv.) | 5035.08 | 5431.25 | 5340.31 | 5357.26 |
| DMPC (as is) | 637.15 | 558.2 | 568.31 | 605.7 |
| DMPC (deriv.) | 634.63 | 552.68 | 535.51 | 582.72 |
| MHP (calc.) | 0.00 | 192.80 | 282.51 | 104.21 |
| CHP (calc.) | 0.00 | 0.00 | 0.00 | 0.00 |

The invention has thus been described with reference to operative examples of the method according to the current invention. It is expected that the method according to the current invention will have application in any process or facility that makes use of or produces organic hydroperoxides. Non-limiting examples of such processes include the production of phenol by cleavage of CHP, polymerization processes utilizing peroxide initiators and controlled rheology processing of polymers. The preceding examples should not be construed as limiting. The full scope of the invention will be made clear by the claims appended hereto.

What is claimed is:

1. A method for determining the concentration of an organic hydroperoxide in an aqueous stream, the method comprising the steps of:
providing an aliquot from an aqueous stream containing at least one organic hydroperoxide,
adding acetic acid to said aliquot,
adding potassium iodide to said aliquot,
sealing said aliquot in a sealable sample container,
heating said sealable sample container at a temperature above about 65° C. and below about 100° C. for a time sufficient to convert said at least one organic hydroperoxide into at least one alcohol and equilibrate a gas and a liquid phase in said sample vial,
extracting a gaseous sample of said aliquot from said sealable sample container,
analyzing said gaseous sample by gas chromatography to obtain a quantified value of said at least one alcohol in said gaseous sample, and
correlating said quantified value of said at least one alcohol in said gaseous sample to a concentration of said at least one organic hydroperoxide in said aqueous stream.

2. The method according to claim 1, wherein said sealable sample container is heated at a temperature of about 80° C. for about 15 minutes.

3. The method according to claim 1, further comprising the step of adding water to said aliquot.

4. The method according to claim 1, wherein said potassium iodide is added as a 50% solution in water.

5. The method according to claim 3, wherein said potassium iodide is added as a 50% solution in water.

6. The method according to claim 5, wherein to every 1 mL of said aliquot, from about 0.60 mL to about 0.85 mL of water is added, from about 0.05 mL to about 0.15 mL of acetic acid is added and up to about 0.25 mL of 50% potassium iodide solution in water is added.

7. The method according to claim 1, wherein said at least one organic hydroperoxide is selected from the group consisting of: methyl hydroperoxide, cumene hydroperoxide, sec-butyl hydroperoxide, t-butyl hydroperoxide and mixtures thereof.

8. The method according to claim 1, wherein said aliquot from an aqueous stream is prepared by diluting a sample from an aqueous process stream with water.

9. The method according to claim 8, wherein said sample from an aqueous process stream is diluted with water at a ratio of 1:10 to 1:100.

10. A method for determining the concentration of an organic hydroperoxide in an aqueous stream, said method comprising the steps of:
in a first sealable sample container, providing a first aliquot of approximately 1 mL from an aqueous stream containing at least one organic hydroperoxide,
adding about 0.75 mL of water to said first aliquot,
adding about 0.10 mL of acetic acid to said first aliquot,
adding about 0.15 mL of 50% potassium iodide in water to said first aliquot,
sealing said first sealable sample container,
heating said first sealable sample container at a temperature of about 80° C. for at least about 15 minutes,
extracting a first gaseous sample of said first aliquot from the headspace of said first sealable sample container,
analyzing said first gaseous sample by gas chromatography to obtain a quantified value of said at least one alcohol in said first gaseous sample, and
correlating said quantified value of said at least one alcohol in said first gaseous sample to the concentration of said at least one organic hydroperoxide in said aqueous stream.

11. The method according to claim 10, further comprising the steps of:
in a second sealable sample container, providing a second aliquot of approximately 2 mL from said aqueous stream,
sealing said second sealable sample container,
heating said second sealable sample container at a temperature of about 80° C. for at least about 15 minutes,
extracting a second gaseous sample of said second aliquot from the headspace of said second sealable sample container,
analyzing said second gaseous sample by gas chromatography to obtain a quantified value of said at least one alcohol in said second gaseous sample.

12. The method according to claim 11, wherein said organic hydroperoxide is methyl hydroperoxide and said alcohol is methanol.

13. A method for determining the concentration of an organic hydroperoxide in an aqueous stream, the method comprising the steps of:
providing an aliquot from an aqueous stream containing at least one organic hydroperoxide,
adding a water soluble carboxylic acid to said aliquot,
adding iodide ion to said aliquot,
sealing said aliquot in a sealable sample container,
heating said sealable sample container at a temperature above about 65° C. and below about 100° C. for a time sufficient to convert said at least one organic hydroperoxide into at least one alcohol and equilibrate a gas and a liquid phase in said sample vial,
extracting a gaseous sample of said aliquot from said sealable sample container,
analyzing said gaseous sample by gas chromatography to obtain a quantified value of said at least one alcohol in said gaseous sample, and
correlating said quantified value of said at least one alcohol in said gaseous sample to a concentration of said at least one organic hydroperoxide in said aqueous stream.
